(19) United States
(12) Patent Application Publication
(10) Pub. No.: US 2013/0110089 A1
(43) Pub. Date: May 2, 2013

(54) APPARATUS FOR GUIDING MEDICAL DEVICES AND RELATED METHODS OF USE

(71) Applicants: Gary Kappel, Acton, MA (US); Paul Smith, Smithfield, RI (US); Paul Barner, Arlington, MA (US); Erin Daly, Nashua, NH (US)

(72) Inventors: Gary Kappel, Acton, MA (US); Paul Smith, Smithfield, RI (US); Paul Barner, Arlington, MA (US); Erin Daly, Nashua, NH (US)

(21) Appl. No.: 13/659,744

(22) Filed: Oct. 24, 2012

Related U.S. Application Data
(60) Provisional application No. 61/552,676, filed on Oct. 28, 2011.

(51) Int. Cl. A61B 19/00

(52) U.S. Cl. 606/1

(57) ABSTRACT

A device and related methods of use for guiding a surgical tool. The device may include a mating section for mating with a portion of a guide tube. An elongate structure may extend from the mating section. In addition, the elongate structure may be configured to transition between a collapsed delivery configuration and an expanded deployed configuration.

This application claims priority to U.S. Provisional Application No. 61/552,676 filed on Oct. 28, 2011, the entire contents of which are incorporated herein by reference.

### FIELD OF THE INVENTION

Embodiments of the present disclosure relate generally to medical devices suitable for use in medical procedures. In particular, embodiments of the present disclosure relate to apparatus for guiding or triangulating a number of medical devices upon a target site during a medical procedure.

### BACKGROUND OF THE INVENTION

Endoscopic surgery, also known as minimally invasive surgery, uses an endoscope, or other similar introduction devices, delivered through a small incision or a natural orifice to perform a surgical procedure at a remote site. Conventionally, endoscopes include one or more fiber optic channels for image collection, and one or more working channels for administration of drugs, suction, and irrigation; or to introduce surgical tools (such as baskets, forceps, scissors, snares, graspers, needles, energy delivery devices, biopsy devices, or brushes) for tissue excision, sampling, or other diagnostic and surgical work. Currently, endoscopic surgeries are preferred over traditional (or open) surgeries as these surgeries greatly reduce recovery time and tissue trauma.

The fact that endoscopic surgery is performed remotely—that is, the surgeon does not have a direct view of the operating field—and that the technique lends itself to very precise procedures, produces some unique problems. A significant issue is the problem of precisely locating the surgical tools or an imaging device relative to the target position in the patient's body. Working with only the visibility provided by the endoscope itself, and achieving an exact location for the surgical tools is a difficult proposition. Prior art methods that rely primarily on positioning the surgical tools by eye are both imprecise and time-consuming. Additionally, current laparoscopic surgical device technologies utilize rigid instruments or flexible shafts, which may actually impede the surgeon's view of the surgical site by being in the line-of-sight. The flexible shaft devices may not have the ability, themselves, to be positioned or manipulated around the surgical sight for the optimized combined angle of sight, access, and work.

Thus, improvements in the design and functionality of minimally invasive tools that facilitate easily locating surgical instruments at a target site are desirable.

### SUMMARY OF THE INVENTION

Embodiments of the present disclosure provide a device for guiding a surgical tool to a target site within the body of a patient using a minimally invasive surgical system.

In accordance with an aspect of the present disclosure, a device for guiding a surgical tool may include a mating section for mating with a portion of a guide tube or scope or another type device. An elongate structure may extend from the mating section. The elongate structure may include a proximal end and a distal end. In addition, the elongate structure may be configured to transition between a collapsed delivery configuration and an expanded deployed configuration.

In various embodiments, the device may include one or more of the following additional features: the elongate structure may include multiple elongate structures; distal ends of each of the elongate structure may be generally directed at a common location; the elongate structure may define a lumen extending between the proximal and distal ends; the elongate structure may be a rail for guiding a surgical tool; a triangulation mechanism may move the elongate structure from the collapsed delivery configuration to the expanded deployment configuration; the inner surface of the mating section may include a first geometric configuration configured to mate with a corresponding second geometric configuration on the distal end of the guide tube; the mating section may be configured to be secured to the distal end of the guide tube through an interference fit; the device may further include a sealing mechanism for preventing the flow of bodily materials into the guide tube; and the elongate structure may be self-expandable.

According to another embodiment, a surgical system may include a proximal portion having a plurality of openings for receiving surgical tools therein. The surgical system also may include a guide tube extending distally from the proximal portion. The guide tube may define a number of channels corresponding to the openings in the proximal portion. In addition, the surgical system may include a distal portion including a multiple openings corresponding to the channels defined by the guide tube. The distal portion may further include at least one elongate structure extending distally from the at least one of the openings on the proximal portion. Each elongate structure may include a proximal end and a distal end. In addition, the elongate structure may be configured to transition between a collapsed delivery configuration and an expanded deployed configuration.

In various embodiments, the surgical system may include one or more of the following additional features: the elongate structure may include a lumen, extending between its proximal and distal ends, configured to receive a surgical tool therein, the elongate structure may include a number of elongate structures, and when in an expanded configuration, the distal ends of each of the elongate structures may generally directed at a common location; the elongate structure may be fixedly secured to the guide tube; the proximal portion may further include a control member for selectively manipulating a positioning of the distal portion; at least one of the elongate structures may include a rail for guiding a surgical tool; the distal portion may be detachably secured to the elongate structure; the surgical system may further include a triangulation mechanism for moving the elongate structures from the collapsed delivery configuration to the expanded deployed configuration; the triangulation mechanism may be expandable; and at least one of the elongate structure may be self-expandable.

A further aspect of the present disclosure includes a method for performing a surgical procedure at a target site of a body. The method may include advancing a surgical system to a location adjacent the target site. The surgical system may include a proximal portion including a plurality of openings for receiving surgical tools therein. The proximal portion may remain outside of the body. The surgical system also may include a guide tube extending distally from the proximal portion. The guide tube may define a plurality of channels corresponding to the openings in the proximal portion. Further, the surgical system may include a distal portion including a plurality of openings corresponding to the channels defined by the guide tube. The distal portion may further include at least one elongate structure extending distally from one of the openings, each elongate structure having a proximal end and a distal end. Each elongate structure may be configured to transition between a collapsed delivery configuration and an expanded deployed configuration. The method may further include transitioning the elongate structure from the collapsed delivery configuration to the expanded deployed configuration. Subsequently, the method may include advancing at least one surgical tool from an opening in the proximal portion and through a channel in the guide tube and opening in the distal portion. The method may also include guiding the at least one surgical tool to a target location via the elongate structure and performing a procedure with the surgical tool.

Additional objects and advantages of the claimed invention will be set forth in part in the description, which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the present disclosure and together with the description, serve to explain the principles of the embodiments disclosed herein.

FIG. 1 is a perspective view of a minimally invasive surgical system, according to an embodiment of the present disclosure.

FIG. 2 is a cross-sectional view of the system shown in FIG. 1, taken along plane A-A.

FIG. 3 is a schematic view of an exemplary surgical instrument for use with the system of FIG. 1.

FIG. 4 is a perspective view of the distal end of the exemplary system shown in FIG. 1, including a guiding mechanism, in accordance with an embodiment of the present disclosure.

FIG. 5 is a schematic view of an alternate embodiment of a guiding mechanism, in accordance with a further embodiment of the present disclosure.

FIGS. 6A and 6B are schematic views of the guiding mechanism shown in FIG. 4, in collapsed and expanded states, respectively.

FIG. 7 is a side view of the distal end of the system shown in FIG. 1, having an expandable balloon connected to the distal end acting as a triangulating mechanism, in accordance with another embodiment of the present disclosure.

FIG. 8 is a side view of the distal end of the system shown in FIG. 1, including an alternate embodiment of the triangulating mechanism including a spring mechanism, in accordance with another embodiment of the present disclosure.

FIG. 9 is a side view of the distal end of the system shown in FIG. 1, including control wires acting as a triangulating mechanism, in accordance with yet another embodiment of the present disclosure.

FIGS. 10A and 10B depict a side view of the distal end of the system shown in FIG. 1, including an alternate embodiment of the triangulating mechanism shown in FIG. 9.

FIG. 11 depicts an embodiment of a triangulating mechanism employing a triangulating guide device, in accordance with an embodiment of the present disclosure.

FIGS. 12A and 12B are side views of the distal end of the system shown in FIG. 1, including an alternate embodiment of a triangulating mechanism.

FIGS. 13A and 13B illustrate embodiments of the present disclosure including a method of using the system to perform a surgical procedure.

FIG. 14 illustrates another embodiment of the present disclosure depicting a sealing mechanism while using the disclosed system to perform a surgical procedure.

### DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts.

### Overview

The present disclosure relates generally to minimally invasive surgical systems, such as endoscopic and laparoscopic systems, having one or more medical devices inserted therein to perform a surgical procedure. The medical devices may include, but are not limited to, optical devices or surgical instruments such as, e.g., forceps, scissors, biopsy devices, needles, tissue manipulators, surgical staplers, implant delivery devices, energy delivery devices, or tissue graspers (hereafter, “tools”). These tools may be configured to move in three dimensions selectively. The desired location for the employment of the tools for performing the planned procedure is referred to here as the “target site.” The target site may include relative position data for a number of tools, including angular orientation. The process of converging the desired set of tools to form a direct, clear vision path, and a working volume to the target site may be referred to as “triangulation.”

Triangulation assists in obtaining a clear view of a target site and permits effective surgical procedure such as, e.g., concomitant retraction and dissection of tissues. To this end, embodiments of the present disclosure employ a guiding mechanism that guides one or more tools to facilitate triangulation upon a target site.

Embodiments of the present disclosure can be used for a variety of diagnostic or therapeutic procedures, including, but not limited to, colonoscopy, upper endoscopy, bronchoscopy, thoracoscopy, laparoscopy, ureteroscopy, hysteroscopy, and video endoscopy. Although exemplary embodiments of the present disclosure are described with reference to endoscopes, it will be appreciated that aspects of the present disclosure may have wide application. These devices and techniques may be suitable for use with other medical devices, where tool convergence toward a surgical location is desirable. Accordingly, the following descriptions and illustrations should be considered illustrative in nature, not limiting the scope of the claimed invention.

### Exemplary Embodiments

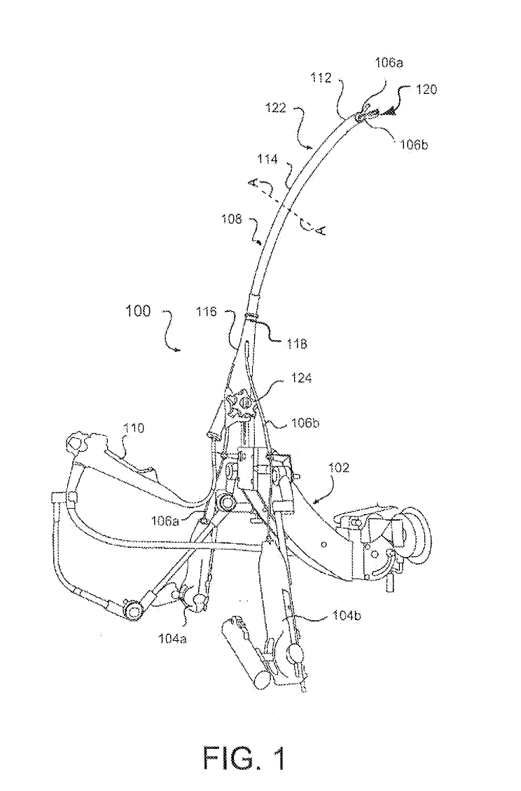

FIG. 1 provides a perspective view of an embodiment of a minimally invasive surgical system, such as an endoscopy system 100, for performing surgeries or other procedures within a patient's body, such as, e.g., intraluminal, percutaneous, and/or transluminal surgery, through an incision, a natural body opening. The system 100 may include a frame 102 for supporting control members 104a and 104b for controlling surgical tools 106a and 106b, respectively. A guide tube 108, extending from the frame 102, houses the elongate bodies (not shown) of tools 106a, 106b, an optical device 110, and/or suitable channels for e.g., irrigation or suction. As used herein, “guide tube” may include, but is not limited to, guide tubes, introducers, endoscopes, catheters, tools with lumens, or other suitable introduction devices. When the guide tube 108 is inserted into a patient, control members 104a, 104b may allow a surgeon to manipulate or otherwise control tools 106a, 106b, which extend to a remote surgical site positioned adjacent to a distal end 112 of the guide tube 108. It should be understood that the frame 102 can be designed with a variety of configurations depending on patient's location, spacing, ergonomics, physician preference, procedure type, and/or the availability of an operating table frame. In addition, although the depicted embodiment of endoscopy system 100 is shown with two tools 106a, 106b, a greater or lesser number of suitable tools may be used with endoscopy system 100.

The guide tube 108 may be inserted through a natural orifice and/or incision to a surgical site within a patient. Guide tube 108 may have an elongate body 114 having a proximal end 116 and the distal end 112. The term “distal” refers to the end of the guide tube 108 farthest away from a medical professional when introducing guide tube 108 in a patient. By contrast, “proximal” refers to the end of guide tube 108 closest to the medical professional when placing guide tube 108 in the patient. Although guide tube 108 may extend from frame 102 in FIG. 1, guide tube 108, however, may be configured for use without the frame 102 during a portion or all of a surgical procedure. The proximal end 116 defines at least one proximal end opening 118, for receiving surgical tools, such as, for example, tools 106a, 106b and/or optical device 110. Further, the distal end 112 may define at least one distal end opening 120 for introducing the tools to a target site within a patient. Between proximal end 116 and distal end 112 of guide tube 108, the elongate body 114 can include a mid-portion 122. In one embodiment, the mid-portion 122 may be generally flexible and non-articulating. In another embodiment, at least a portion of the guide tube 108 may be rigid. For example, a portion, various portions, or the complete guide tube 108 can be rigid or selectively rigid.

In one embodiment, guide tube 108 can be articulable in one or more degrees of freedom. For example, guide tube 108 can be articulated with controls 124 to move at least a portion of guide tube 108 (e.g., distal end 112) up/down and/or side-to-side. Additional degrees of freedom, provided for example via, rotation and/or translational movement of the guide tube 108 with respect to the frame 102, or additional articulating bending sections, are also contemplated. In such an embodiment, endoscopy system 100 may be provided with suitable steering systems for, among other things, articulating and/or steering distal end 112 of guide tube 108. For example, a suitable steering system may include one or more of pulleys, control wires, gearing, electrical actuators (e.g., servomotors), pneumatic actuators, and the like.

The outer surface of elongate body 114 may include any suitable coating and/or covering. For example, an outer surface of elongate body 114 may include a layer of lubricous material to facilitate insertion of guide tube 108 through a body lumen or surgical insertion. As alluded to above, elongate body 114 may define one or more channels adapted to guide at least one elongate tool to a surgical site. The surfaces of the channels within elongate body 114 may also include any suitable coating or covering. For example, the surfaces of the channels may include a similar lubricious coating. In addition, a portion of the channels within elongate body 114 may include one or more suitable sealing mechanisms (not shown). The sealing mechanisms may serve to inhibit or prevent the proximal movement of bodily fluids and/or materials into and through guide tube 108. Likewise, the sealing mechanisms may help prevent the insertion of unwanted materials through guide tube 108. In one embodiment, a suitable sealing mechanism may include an elastomeric barrier having an expandable and resealable opening therethrough.

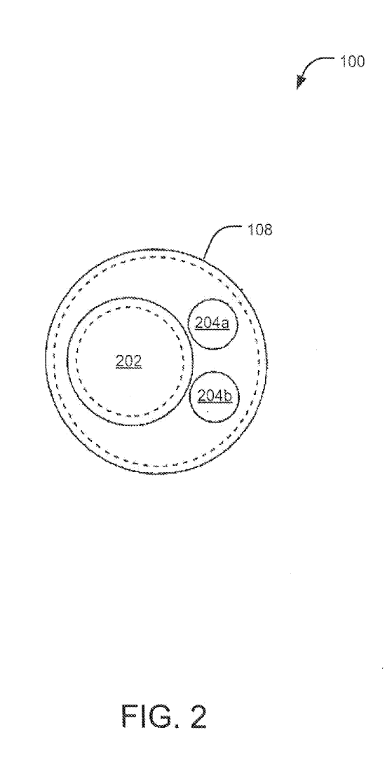

FIG. 2 illustrates an exemplary cross-sectional view of a portion of the elongate body 114 (taken along line A-A in FIG. 1) that includes a main channel 202 and working channels 204a, 204b, collectively referred to as channels. In general, the channels can be defined by elongate hollow lumens that extend, at least partially, between proximal end 116 and distal end 112 of guide tube 108. While three channels are illustrated, fewer channels or more channels are also contemplated. The number of channels and their configuration can be varied depending on the intended use of the system and the tools required during a procedure. For example, the guide tube 108 can include a single channel adapted to receive multiple tools or multiple channels for multiple instruments. In addition, while the main channel 202 is described as the largest channel, in terms of cross-sectional width, the working channels 204a, 204b can be of larger or smaller size than the main channel 202. Further, channels 202, 204a, 204b, of guide tube 108 may have any suitable cross-sectional configuration. For example, as shown, each of channels 202, 204a, 204b may have a substantially circular cross-sectional configuration. In some embodiments, however, one or more of channels 202, 204a, and 204b may have a non-circular cross-sectional configuration that corresponds to a configuration of a particular tool. In this manner, a particular channel may be specifically designed and dedicated for particular tools. In addition, although the depicted embodiments illustrate that channels 202, 204a, and 204b include substantially uniform cross-sectional configurations and dimensions, the cross-sectional dimensions and/or configurations of any of channels 202, 204a, and 204b may be varied along its length, as desired. For example, a proximal portion of channel 202 may include a hexagonal cross-sectional configuration while a distal portion of channel 202 includes a rectangular cross-sectional configuration.

Moreover, use of the word “channel” does not require that the optical devices 110 and/or tools 106a, 106b traversing the guide tube 108 be distinct or stand-alone devices. For example, in one embodiment, endoscope system 100 may include an optical device and/or tools formed integrally with the guide tube 108. In still another embodiment, the optical devices and/or tools may themselves define the guide tube 108. For example, the optical device 110 can define guide tube 108 and include channels for tools. Channels may also be located on the outside of guide tube 108.

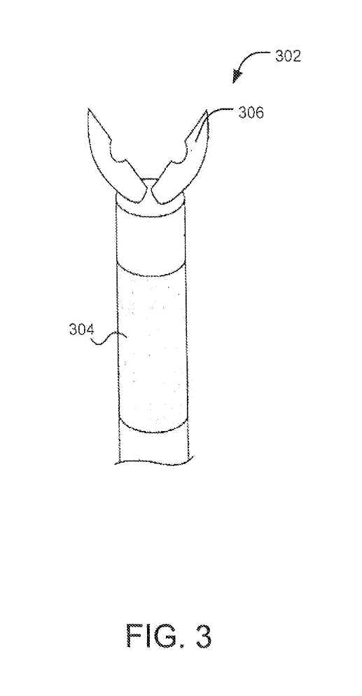

Each channel may allow passage of one or more tools to perform a desired procedure. The tools may include optical devices, or other tools such as jaws, hollow tubes, hooks, barbs, needles, or other tissue manipulation instruments known in the art, as already discussed above. FIG. 3 illustrates an exemplary tool 302 having an elongate body 304 and a distal end-effector 306, which in this case, is a pair of jaws. The tool 302 may be configured for insertion into a patient through a guide tube, such as, e.g., guide tube 108. For example, tool 302 may extend from proximal end 116 to distal end 112 of guide tube 108 (FIG. 1) within one of the channels (such as working channels 204a). Moreover, the distal end-effector 306 may extend out of distal end 112 of the guide tube 108 towards the target site to perform a surgical procedure such as grasping, sampling, or otherwise manipulating tissues. In addition, actuation of the distal end-effector 306 may be controlled by any suitable means known in the art, including, e.g., a control member (not shown) disposed adjacent proximal end 116 of the guide tube 108. Exemplary tools may include, but are not limited to, graspers, dissectors, irrigation/aspiration tubes, needles, and/or J-hook cautery devices.

Since the channels within guide tube 108 generally extend parallel to one another, tools 106a, 106b, when inserted through guide tube 108, also extend from guide tube 108 generally parallel to each other. In some cases, inserting tools 106a, 106b in such a manner may make it difficult to use tools 106a, 106b cooperatively or consecutively. That is, because tools 106a, 106b extend generally parallel to one another, it may be difficult to manipulate tools 106a, 106b to have their respective end-effectors simultaneously converge upon a desired target site. Thus, it may be necessary to facilitate “triangulation” of tools 106a, 106b. Triangulation includes directing surgical tools into a more precise alignment and positioning within the patient's body to perform a specific procedure. In general, tools can assume a desired angular position to converge to a point to form a triangular arrangement, each tool pointing towards the target site. If a given procedure requires tools to diverge, that alignment could be achieved as well. Achieving this triangulation has proved extremely difficult using conventional tool manipulation techniques. To overcome this issue, the embodiments of the present disclosure employ a guiding mechanism disposed at or proximate a distal end of the guide tube 108 to accomplish triangulation of tools.

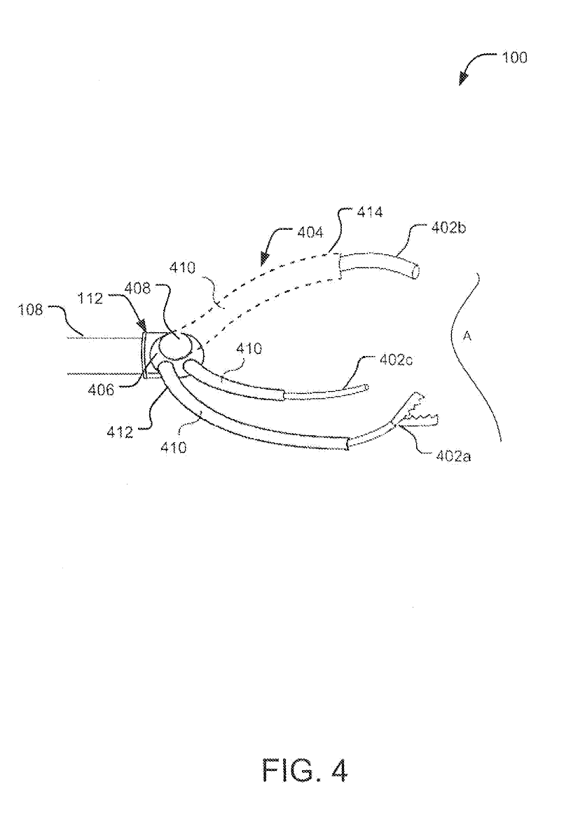

The following sections describe different embodiments of a guiding mechanism of the present disclosure. FIG. 4 is a schematic view of distal end 112 of the guide tube 108 including an embodiment of guiding mechanism, in accordance with the principles of the present disclosure. The guide tube 108 may include three channels (not shown) for delivering tools, such as grasper 402a, an optical device 402b, and a hollow tube 402c, (hereafter, “tools 402”). A guiding structure 404, disposed at distal end 112 of the guide tube 108, may assist in triangulating the tools 402 upon a desired target site “A”. The guiding structure 404 may include an end cap 406 that may include a mating section for attachment with distal end 112 of guide tube. It should be understood that various alternatives of the mating section may be contemplated. The following section describes some of the mating sections.

As shown, end cap 406 may be a flat circular member, e.g., a plate, that substantially covers distal end opening 120 of the guide tube 108. In an alternative embodiment, end cap 406 may include a ring or ring-like configuration acting as mating section for securing the cap 406 to a distal end of guide tube 108. Alternatively, end cap 406 may include a ring configuration that acts as a flange, and one side of the ring includes a substantially flat surface. In such a configuration, the ring and the flat surface may define a cavity, which may receive distal end 112 of guide tube 108. As shown, end cap 406 may include a circular cross-sectional configuration having a diameter equal to or greater than the diameter of distal end 112 of the guide tube 108 to secure the end cap 406 to distal end 112. In general, end cap 406 may include a cross-sectional configuration substantially similar to the cross-sectional configuration of distal end 112 of the guide tube 108. Alternatively, end cap 406 or portions of end cap 406 may include any desired cross-sectional configuration such as a hexagonal or square cross-section.

The diameter of the end cap 406 may be either large enough such that end cap 406 attaches to distal end opening 120 of the guide tube 108 through any known attachment means including snap fit, threads, adhesives, or other known sealing mechanisms. For example, the inner surface of the end cap 406 having a ring configuration and distal end 112 of the guide tube 108 may include corresponding threads that may provide attachment between the two parts. In some embodiments, end cap 406 may include additional geometrical structures, such as projections or keyed structures, allowing the cap 406 to attach with the guide tube 108. In such embodiments, these geometrical structures may fit snugly into corresponding grooves, notches, or indentations formed on distal end 112 of the guide tube 108. The geometrical structures may also facilitate correct orientation of end cap 406 relative to guide tube 108. End cap 406 may either receive a distal end 112 of endoscope system 100, or a lip of the endoscope system 100. End cap 406 may be either permanently or detachably connected to the guide tube 108. In addition, one or more features of end cap 406 may be made integrally with, a portion of guide tube 108. Further, in some embodiments, one of end cap 406 or guide tube 108 may include a sealing mechanism to seal the two components relative to one another, thereby preventing, e.g., possible leakage of bodily fluids.

Furthermore, end cap 406 may include multiple openings 408 for receiving tools 402. For example, the flat surface of the end cap 406 may include openings 408 for receiving at least one surgical tool. Alternatively, when the end cap 406 includes a ring configuration, a number of additional smaller diameter ring structures may be connected to the inner circumference of the ring. These additional rings receive surgical tools disposed within the channels 202, 204a, and 204b. As shown, the number of openings in the end cap 406 may correspond to the number of channels, such that each opening receives a single tool. Those skilled in the art, however, will appreciate that the number of openings may not be equal to the number of channels such that an opening 408 may receive more than one tool from one or more channels.

Further, guiding structure 404 may include a set of hollow tubular members 410, each having a proximal end 412, a distal end 414, and a lumen extending therebetween, extending outward from each of openings 408. Distal ends 414 of the tubular members 410 may be designed to reduce trauma and irritation to surrounding tissues. Distal ends 414 may include rounded or beveled terminal ends and/or faces, for example.

As shown, the tubular members 410 may be elongate structures providing passage for one or more tools 402 to pass from the channels 202, 204a, and 204b into a working space within a patient. It should be understood that tools 402 follow the contour of the tubular members 410 and exit the distal ends 414 of the tubular members 410 at generally the same angle as the tubular members 410 with respect to the guide tube 108. For example, a tool advancing through a curved lumen exits the lumen at about a 30 degree angle with respect to the longitudinal axis. In other embodiments, this angle may be greater or less than 30 degrees. Consequently, the tools 402 are caused to converge upon a target site.

The angular orientations of the tubular members 410 assist in guiding or triangulating tools 402 to the target site “A”. In general, tubular members 410 have an angled configuration relative to the longitudinal axis of the guide tube 108 such that tools 402 may diverge or converge as they exit the distal end of the guide tube 108. In an embodiment, articulating or pre-curved tools 402 may also be delivered through tubular members 410. The angle of deflection of tools 402 in combination with the divergence angle of tubular member 410 may provide additional optionally controllable divergence or convergence for tools 402.

In the depicted embodiment, each tubular member 410 initially diverges away from the other of tubular members 410 and then converges towards one another. As a result, each tool 402 extending out from the tubular members 410 points towards a particular location (e.g., target site “A”) illustrating triangulation of tools 402. As shown, tubular members 410 form an arc-shaped structure that provides a converging passageway that not only directs the distal end-effectors of tools 402 to a particular location in the body, but also spaces the distal end of the tools 402 from one another within a body cavity. The increased spacing between the surgical tools 402 may increase the volume of the area in which the surgical tools can work (or work with one another).

The tubular members 410 may be formed using any of a variety of different geometries and configurations. For example, each of tubular members 410 may have varying radii of curvature based on the target site. Further, the number of tubular members 410 extending outward from the end cap 406 may vary in number. The illustrated embodiment depicts only three tubular members 410 for purposes of description. As alluded above, however, the number of tubular members 410 extending away from end cap 406 may correspond to the number of channels disposed within guide tube 108. In an embodiment, only one tubular member 410 may extend out from end cap 406, which may include a single opening 408 to accommodate one tubular member 410. In addition, the diameter of the tubular members 410 may be substantially large enough to allow passage of varying sized tools. It should be understood that the diameter and length of the tubular members 410 may be tailored to the individual needs of particular patients.

Tubular members 410 may have any suitable cross-sectional configuration. As shown, each of the tubular members 410 may have a substantially circular cross-sectional configuration. In some embodiments, however, one or more of tubular members 410 channels may have a cross-sectional configuration that corresponds to the configuration of a particular channel such as the channels 202, 204a, 204b. In addition, although the depicted embodiment illustrates that tubular members 410 include substantially uniform cross-sectional configurations and dimensions, the cross-sectional dimensions and/or configurations of any of the tubular members 410 may be varied as desired. In addition, the cross-sectional dimensions and/or configurations of any of the tubular members 410 may be varied along the length of tubular members 410 as desired. For example, one tubular member 410 may have a circular cross-sectional configuration while other may have a hexagonal cross-sectional configuration. Further, the cross-sectional configuration of one or more tubular members 410 may be rectangular at one end and hexagonal at the other, for example.

In another embodiment, only one of tubular members 410 may be angled while the remaining tubular members 410 extend parallel to the longitudinal axis of the guide, tube 108. Based on that configuration, two tools may be deployed straight out from the guide tube 108, and the third tool may be angled downward, so that the third tool may be triangulated upon the location of the other two tools. In still other embodiments, more than one or all of the tubular members 410 can extend in an arcuate configuration. Further, the angle at which each tool 402 exits the tubular members 410 need not be the same. Additionally, not only can the curved tubular members 410 direct the tools 402 exiting the guide tube 108 in a predetermined manner, but they can also serve to maintain some predetermined spacing between the distal ends of the tools 402 while the system 100 is in use. For example, the curvature of the tubular members 410 ensures that the tools exiting the tubular members 410 are adequately spaced within the working area of the patient.

The outer surface of elongate body 114 may include any suitable coating and/or covering. For example, an outer surface of elongate body 114 may include a layer of lubricous material to facilitate insertion of tubular member 410 through a body lumen or surgical insertion. In addition, the inner surface of the tubular member 410 may also include a lubricous coating that allows convenient passage for incoming tools. Furthermore, radiopaque or sonoreflective markings (not shown) may be added to an exterior surface of the tubular member 410. These markings facilitate detection of a position and/or orientation of the tubular member 410 within the patient's body, and a surgeon, with the aid of suitable imaging equipment, may track the path followed by the endoscope system and avoid potential damage to sensitive tissues.

Tubular members 410 may be made of any suitable material that is compatible with living tissue or a living system, non-toxic or non-injurious, and does not cause immunological reaction or rejection. Such materials may include nitinol, ePTFE, fabric, and suitable nickel and titanium alloys. In some embodiments, tubular members 410 may be fabricated using shape memory material wires that may be woven or braided together. Shape memory material, such as nitinol, allows transition of the tubular members 410 from one shape to another upon exposure to a trigger, such as, provided heat, body heat, and/or chemistry.

Tubular members 410 may be made of resilient materials that expand, from a collapsed configuration, when a tool is inserted through them. For example, the tubular members 410 could expand to fit snuggly around a particular tool, thereby providing a seal against the ingress of body fluids and materials. Similarly, tubular members 410 should be flexible enough to permit transitioning between collapsed and expanded configurations.

In addition, in an embodiment, tubular members 410 may be made of discrete sections movably linked to one another. Each section may move relative to the adjacent sections, imparting flexibility to the tubular members 410. Discrete sections may be made of a variety of materials including metal, non-metal, or shape memory metal such as Nitinol. In addition, the direction or position of any of tubular members 410 discrete sections may be selectively and/or temporarily fixed or locked, using known locking mechanism, to provide a desired shape to the tubular member 410.

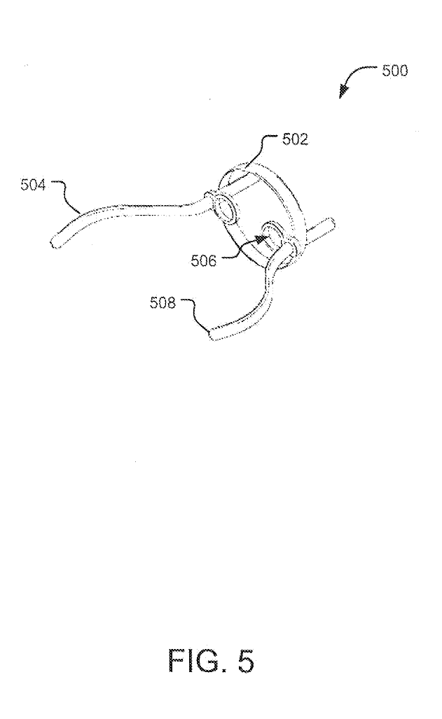

FIG. 5 illustrates an alternate guiding structure 500 including an end cap 502 configured to be disposed at distal end 112 of the guide tube 108 (not shown), and a plurality of rails 504 extending from end cap 502. Although the depicted embodiment illustrates two rails 504 extending from end cap 502, a greater or lesser number of rails 504 may extend from end cap 502. For example, the number of rails 504 provided on end cap 502 may correspond to the number of channels defined by guide tube 108. In addition, end cap 502 may include a set of openings 506 for receiving tools (not shown), with rails 504 extending outward from a location proximate to openings 506 such that the tools received through the openings 506 follow the contour of the rails 504. Those skilled in the art will appreciate that tools or rails 504 may include known mechanism that allows movement of tools on rails 504. For example, rails 504 may include ring-like configurations, curved barbs, grooves, or similar geometrical structure disposed along the length of the rails 504 to maneuver the tools to follow the contour of the rails. These geometrical structures may be disposed at predetermined gaps along the length of the rails. Rails 504 and tubular members 410 have features that allow tools, such as tools 402, to securely traverse along rails 504 or within tubular members 410, without detaching.

In general, the rails 504 may be curved elongate structures having a distal end 508 pointing towards the target site. To facilitate triangulation of tools, each rail 504 may assume an angular orientation that allow the tools traversing on them to converge towards a target site. Those skilled in the art will understand that rails 504 may be made of a variety of materials including metals, non-metals, polymers, or shape memory materials such as Nitinol.

Rails 504 may have a substantially circular cross-sectional configuration. In addition, although the depicted embodiment illustrates that rails 504 include substantially uniform cross-sectional configurations and dimensions, the cross-sectional dimensions of any of the rails 504 may be varied as desired. In addition, distal ends 508 of the rails 504 may include rounded or flared terminal ends and/or faces. These design modifications may reduce trauma and irritation to surrounding sensitive tissues. The surface of rails 504 may include any suitable coating and/or covering, such as a layer of lubricous material to facilitate insertion of rails 504 through a body lumen or surgical insertion. In some embodiments, rails 504 may also include lumens for delivering drugs, tools, and irrigation/aspiration. The outer surface of the tools and rails 504 may be coated with a lubricous coating to enable smooth movement of one over the other. Further, one or more of rails 504 may be selectively extendable or deployable from e.g., end cap 502, as explained in greater detail below.

The bent shape of the tubular members 410 and/or rails 504 may impose a risk of damaging sensitive tissue locations while insertion, and may require a wider body lumen to pass through. To this end, tubular members 410 and rails 504 may be flexible and/or may transition between a collapsed configuration and an expanded configuration. The tubular members 410 and/or rails 504 may be provided in a collapsed state, where members 410 and rails do not triangulate, and once deployed, the members 410 and/or rails 504 may expand using any known expansion mechanism. Alternatively, the tubular members 410 and/or rails 504 may be compressed by applying tension during insertion and once deployed, the tubular members 410 and/or rails 504 may return to their original, expanded configuration. The following sections discuss the two possible configurations of the tubular members 410 and the rails 504. The illustrations, however, only depict the tubular members 410 in a collapsed and an expanded configuration, for illustration purposes.

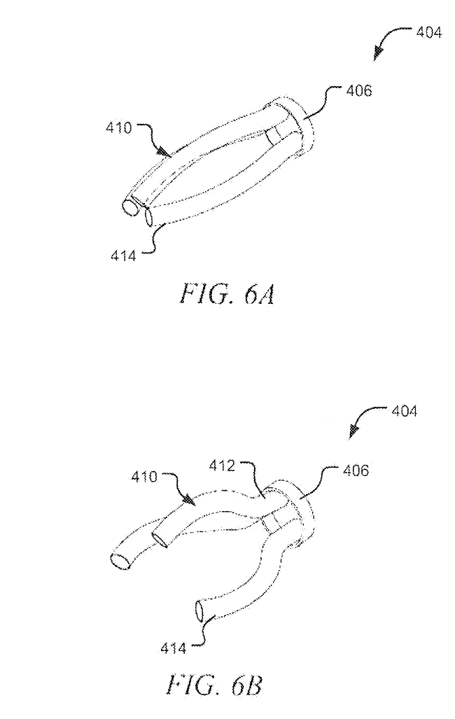

FIGS. 6A and 6B illustrate a collapsed and an expanded state of the tubular members 410, respectively. As shown, in the collapsed state, the tubular members 410 may extend in planes that are substantially parallel to the longitudinal axis of the guide tube 108. In addition, a radius of curvature of tubular members 410/rails 504 in the collapsed state may be smaller than when in the expanded state. The endoscopy system 100 may be inserted into the patient's body with tubular members 410 and/or rails 504 in the collapsed state, which allows convenient navigation through tortuous body anatomy.

Once distal end 112 of the guide tube 108 is proximate to a target site tubular members 410/rails 504 may expand to the expanded state, as shown in FIGS. 5 and 6B. As shown, the expanded state may be substantially arch-shaped such that the distal ends of members 410/rails 504 may be triangulated toward a target site. Tools exiting the distal ends of the tubular members 410 in the collapsed state may be substantially parallel to each other. Expansion of the tubular members 410 may allow the tools to be angularly positioned such that distal ends of each tool converge towards a target site. This convergence facilitates, among other things, appropriate viewing of the target site through the optical device and a greater working space for the tools to perform the desired procedure.

In an alternate embodiment, the members 410 may assume in any desired configuration such as one or more tubular members 410 may diverge from the parallel configuration. Certain target locations may be reached by diverging the tubular members 410 instead of convergence, as discussed above. Accordingly, some or all the members 410 and/or rails 504 may diverge in a desired direction. Subsequently, the tools following the contour of these diverging elements may also diverge in the same direction.

The tubular members 410/rails 504 may exhibit the states depicted in FIGS. 6A and 6B using any suitable mechanisms known to those skilled in the art, including, but not limited to, those described below. Such mechanisms may cause tubular members 410/rails 504 to converge towards a target site. Each tubular member 410 may be independently controlled to triangulate in a desired direction using one or more suitable triangulation means.

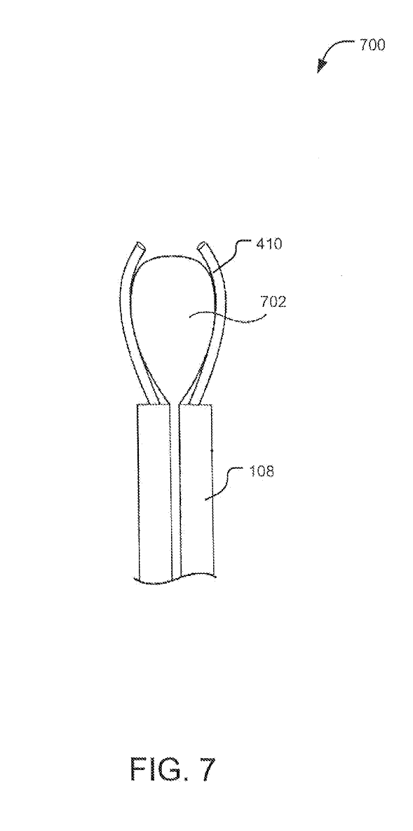

FIG. 7 is a side view of distal end 112 of guide tube 108 including an embodiment 700 of a triangulation mechanism. The triangulation mechanism may include an expandable device 702, such as, for example, an inflatable balloon or expandable basket, positioned between the tubular members 410 such that each tubular member 410 may come into contact with device 702 as device 702 expands. The tubular members 410 may be designed to be substantially parallel to each other, as shown in FIG. 6A. Thus, the tubular members 410 remain in the collapsed state during insertion into a patient's body. In one embodiment, the tubular members 410 may be first positioned within the body at a desired location and subsequently, tools, such as tools 402, may be introduced. Once the tools (not shown) have been delivered to a desired location, the device 702 may be selectively expanded, thereby moving tubular members 410 to the expanded configuration.

The device 702 may be inflated to expand the tubular members 410 by supplying suitable inflation fluids. For example, the device 702 may be expanded by supplying air or saline through an inflation lumen. Those skilled in the art will comprehend that any suitable expansion device, such as a spring, basket, or coil structure, may also be employed. Moreover, these expansion devices may also be connected to the rails 504 to enable transition between a collapsed and an expanded configuration of the rails 504. In addition, the expansion devices may be actuated or self-expandable.

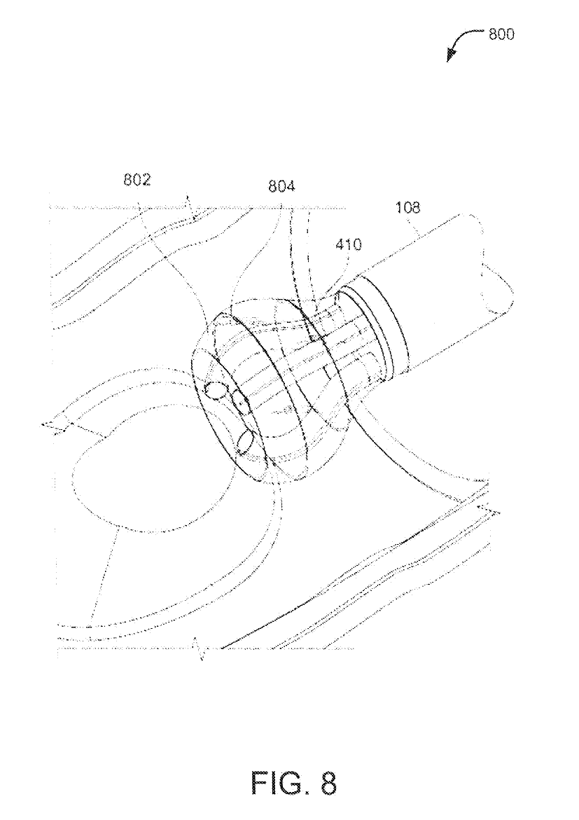

FIG. 8 depicts another embodiment 800 of a triangulation mechanism employing a spring mechanism to expand tubular members 410 from their compressed state. As shown, the triangulation mechanism utilizes a sheath 802, wrapped around one or more springs 804, which encloses tubular members 410. Springs 804 may be connected to a pull cord (not shown) that may extend along the length of guide tube 108 to the proximal end 116. In general, spring 804s along with sheath 802 compresses tubular members 410 in a collapsed state for insertion into a patient's body. Springs 804 may be in their tensioned states at this stage. Once the tubular members 410 reach the desired location, the pull cord is released, allowing springs 804 to expand, thereby causing tubular members 410 to reach their deployed positions. Further, although the depicted embodiment shows springs 804 disposed about the tubular members 410, one or more springs 804 may be disposed within the spaced bounded by tubular members 410.

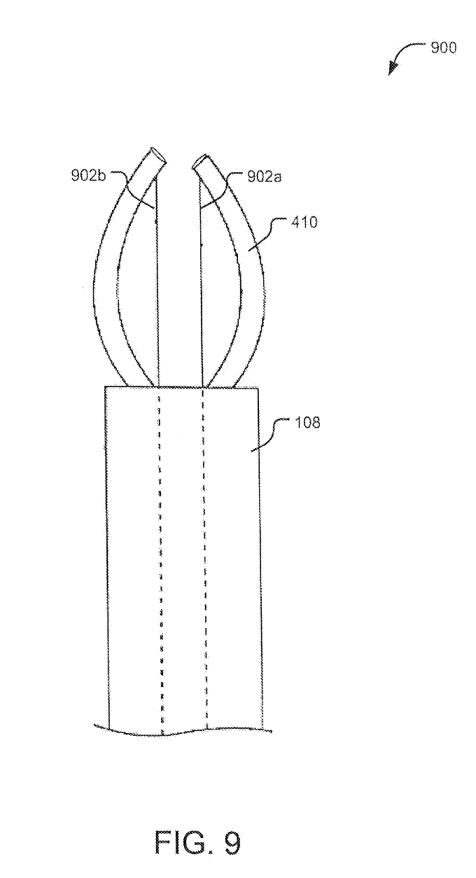

FIG. 9 illustrates an embodiment 900 of distal end 112 of guide tube 108 including control wires 902a, 902b as the triangulation mechanism. As shown, a portion (e.g., distal ends) of tubular members 410 may be connected to the control wires 902a, 902b, which may extend proximally out of a patient's body. Wires 902a, 902b may be configured to selectively maneuver the bend angle or orientation of tubular members 410. To this end, tensioning wires 902a, 902b may cause the tubular members 410 to bow and increase their convergence, resulting in appropriately guiding the tools disposed therein. The tubular members 410 and/or rails 504 may advance into the patient's body in a collapsed state, and once deployed at a desired position, the control wires 902a, 902b may accomplish triangulation of the tubular members 410 and/or rails 504. For descriptive purposes, FIG. 9 depicts two control wires 902a, 902b; it should be apparent, however, that each tubular member 410 and/or rails 504 may be connected to one or more control wires. Further, the angle of convergence of each tubular member 410 and/or rail 504 may be controlled separately by manipulating each control wire independently. For example, in one embodiment, each tubular member 410 may have three or four control wires (not shown) depending therefrom. The control wires may provide for selectively positioning portions of a tubular member 410 relative to other portions of the same tubular member 410.

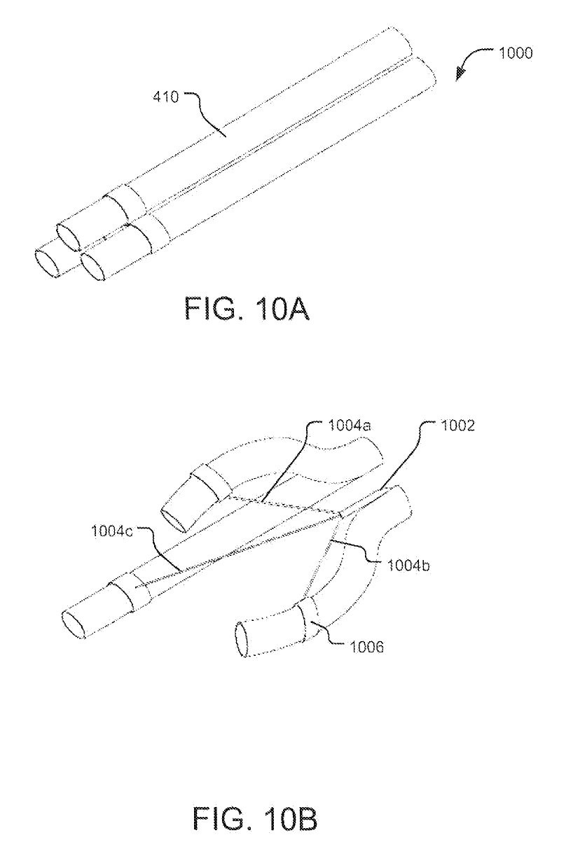

FIGS. 10A and 10B illustrate an alternate embodiment 1000 of the distal end of guide tube 108. Here, instead of manually controlling each control wire, the illustrated embodiment employs a single pull cord 1002 that connects to each tubular member 410 via cords 1004a, 1004b, and 1004c, and extends up to proximal end of guide tube 108. Cords 1004a, 1004b, and 1004c may be connected to the tubular members 410 using a ring-shaped loop (e.g., a collar) 1006 that fits onto each of the tubular members 410. Other attachment mechanisms such as snap fit, direct attachment, and soldering are also contemplated. During insertion, pull cord 1002 may be in a relaxed state, allowing the tubular members 410 to be parallel to each other as shown in FIG. 10A.

Once deployed, pull cord 1002 is activated (e.g., tensioned) to spread the tubular members 410 in a triangulating fashion, as shown in FIG. 10B. To this end, pull cord 1002 may either be connected to a spring that keeps the pull cord tensioned, maintaining the tubular members 410 in the compressed state (FIG. 10A). Once the pull cord 1002 is released, the tubular member 410 expands. In another embodiment, pull cord 1002 along with the cords 1004a, 1004b, 1004c may form an umbrella type mechanism, where pushing the cord 1002 towards the distal end of guide tube 108 triangulates the tubular member 410. In such implementations, the distal end of the guide tube 108 may include a push button to release the pull cord 1002. In each of the above embodiments, pull cord 1002 can be retracted to pull back the spring or the umbrella mechanism and vary the angle of triangulation of tubular members 410. In addition, the embodiment of FIGS. 10A-10B may include any suitable locking mechanism for maintaining tubular members 410 in their deployed states.

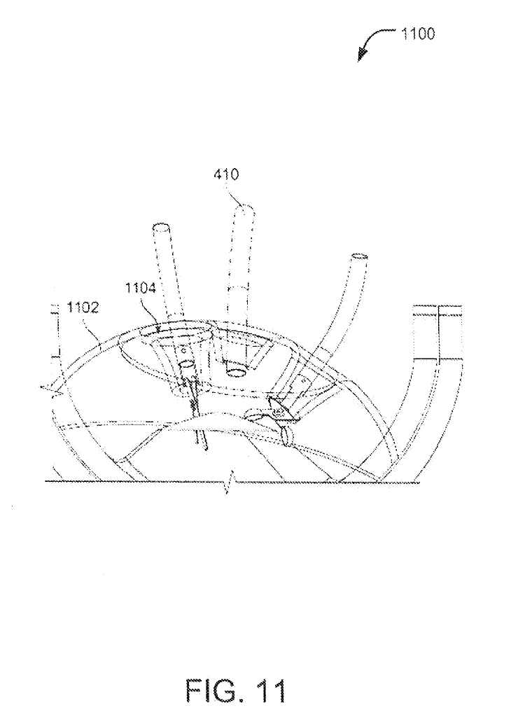

FIG. 11 illustrates an embodiment 1100 of a triangulation mechanism employing a triangulating guide device 1102 to guide the tubular members 410 to a surgical site. In an embodiment, the triangulation guide device 1102 is an expandable member, such as a balloon, having apertures 1104 that are designed to converge towards the surgical site. The number of apertures varies according to the number of tubular members 410 and the angle of convergence may also vary, as desired. In addition, the size of apertures 1104 may be equal or greater than that of the tubular members 410. In an embodiment, the apertures 1104 may be flexible and expandable, allowing tubular members 410 of any configuration to pass through. In addition, the internal surface of apertures 1104 may include known mechanism configured to form a seal around the tubular members 410 and/or rails 504.

In use, the triangulating guide device 1102, in its collapsed form, is inserted to the surgical site and deployed. For deployment, the device may securely position itself around the surgical site using know mechanism. For example, the outer surface of the device 1102 may include glue or gel like material that stick to the body tissues. Alternatively, the outer surface may include hooks or projections that snap onto adjacent tissues to secure the device 1102.

Subsequently, the guide device 1102 expands through external supply of air or fluid, or it may be self-expandable. Alternatively, the triangulating guide device 1102 may be manufactured using shape memory material that expands on exposure to a trigger such as heat or body chemistry. Expanding the triangulation guide device 1102 increases the size of the apertures, providing a working space for tools. With expansion, the apertures assume their desired shape, pointing towards the surgical site, as shown. Next, the tubular members 410 along with tools may advance into the apertures 1104 to triangulate towards the surgical site. The device may include additional integrated tools or device that may assist in entire procedure such as light source, cameras, etc.

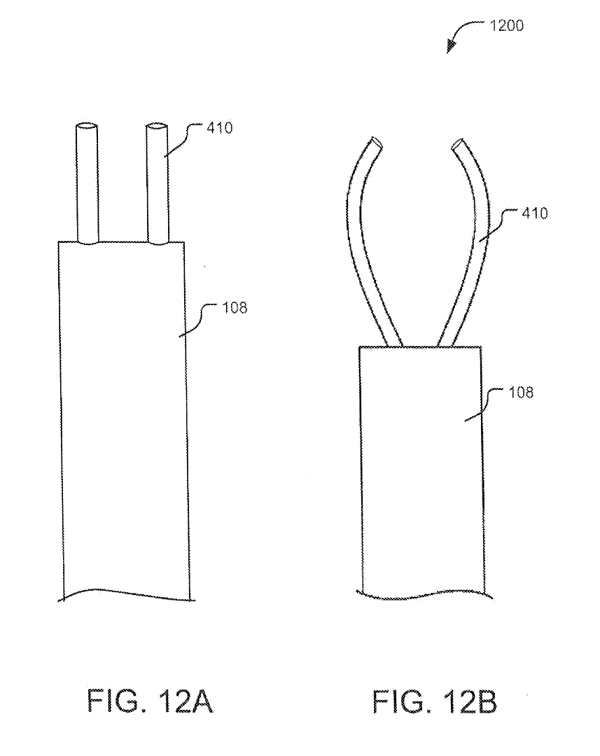

In still another embodiment 1200, as alluded to above, tubular members 410 and/or rails 504 may be fabricated using a shape memory material, such as, e.g., Nitinol. Tubular members 410 may be modified into substantially parallel structures while advancing the endoscope system 100 into the patient's body, as shown in FIG. 12A. The tubular members 410 may be deformed into the collapsed state by applying, for example, heat. Once the distal ends of the tubular members 410 are appropriately positioned within the body, the tubular members 410 may transition into a bent position (as shown in FIG. 12B) after being exposed to a trigger such as, e.g., body heat or chemistry. In some embodiments, external heat may be provided, for example, in the form of heated fluids, resistance heating and/or radiative heating. The tubular members 410 may be designed in a predetermined curved shape and, for the insertion purposes, the tubular members 410 may be deformed into a collapsed state.

In an alternate embodiment, the tubular members 410 and/or rails 504 may be surrounded by a sheath (not shown) that maintains a collapsed state of these elements. Once the tubular member 410 and/or rails 504 are appropriately positioned, the sheath may be retracted proximally and the enclosed elements may transition to their original expanded configuration, as shown in FIG. 12B.

Another mechanism that allows transition of tubular members 410 and/or rails 504 includes movement of these elements within the tube 108 or tubular channels 202. In this configuration, instead of the tubular member 410 and/or rails 504 being fixedly attached to the end cap, these elements may be retractably connected. For example, inner surface of the guide tube 108 or channel 202 may include rails that allow movement of elements 202 and/or 504 within the guide tube 108. Other known retractable attachment mechanism, such as magnetic connection, snap fit connection, may be contemplated. Using any known retractable attachment mechanism, the elements 202 and/or 504 may remain within the guide tube during insertion or retraction process. Once the tube 108 is appropriately deployed, these elements may extend from distal end of the tube 108 towards the desired location. In addition, as these elements extend out, they may expand and converge to a common location.

In some embodiments, the tubular members 410 and/or rails 504 may be made of self-expandable material that transitions from a collapsed state to an expanded state, once deployed. Those skilled in the art will comprehend that the expansion mechanisms discussed in relation to FIGS. 7-12 may be applicable to embodiments of the present disclosure employing rails 504.

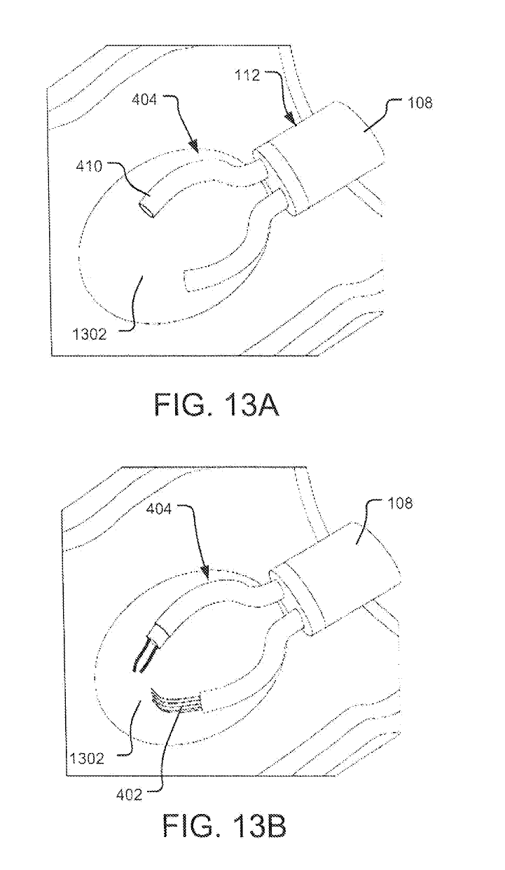

FIGS. 13A-B depict embodiments of the present disclosure including a method of using the system 100 to perform a surgical procedure. FIG. 13A depicts distal end 112 of the guide tube 108 connected to the guiding structure 404. In some embodiments, guiding structure 404 may be an integral part of guide tube 108. Guide tube 108 may be inserted into a patient's body through a small incision or a natural lumen and advanced to a target site. For the purposes of discussion, the described embodiment will aim to remove unwanted tissue, such as a tissue 1302, from the body. To this end, guide tube 108 may be advanced into the body until the guiding structure 404 reaches a location proximate tissue 1302 (i.e., the target site), as shown in FIG. 13A.

In an embodiment of the present disclosure, guide tube 108 connected to the guiding structure 404 may be inserted into the body with tubular members 410 in a collapsed state. Subsequently, tubular members 410 may be manipulated to assume an expanded state to facilitate triangulation of tools passing through the tubular members 410. As discussed above, the manipulation may include appropriately positioning and orienting tubular members 410 such that the distal ends of tubular members 410 point towards the target site 1302. Alternatively, tubular members 410 may be positioned so that they diverge relative to one another. FIGS. 13A-B illustrates the method of using the system 100 with tubular members 410 acting as the guiding structure. Those skilled in the art, however, will understand that the description set out below may be applicable to rails 504 as well.

Tools, such as tools 402, advance into the body through the guide tube 108, extending outward from one of the tubular members 410, as shown in FIG. 13B. The tools 402 traverse along the contour of tubular members 410 to reach out to the body tissue 1302. Due to the curved, converging shape of tubular members 410, the tools 402 triangulate towards target tissue 1302. As tools 402 are guided towards tissue 1302, the desired surgical procedure may be efficiently performed. Moreover, the triangulation of the tools 402 results in providing adequate space for each tool to operate at tissue 1302. FIG. 13B depicts only two tubular members 410 and consequently, two extended tools, for purposes of illustration. Those skilled in the art, however, will understand that the number of tubular members and the tools extending out from the distal end of the guide tube may vary based on the intended use. In a further embodiment, for example, a plurality of tools 402 may be advanced through working channels of elongate member 108. However, guiding structure 404 may only include one tubular member 410, and only one tool may be advanced through that tubular member 410.

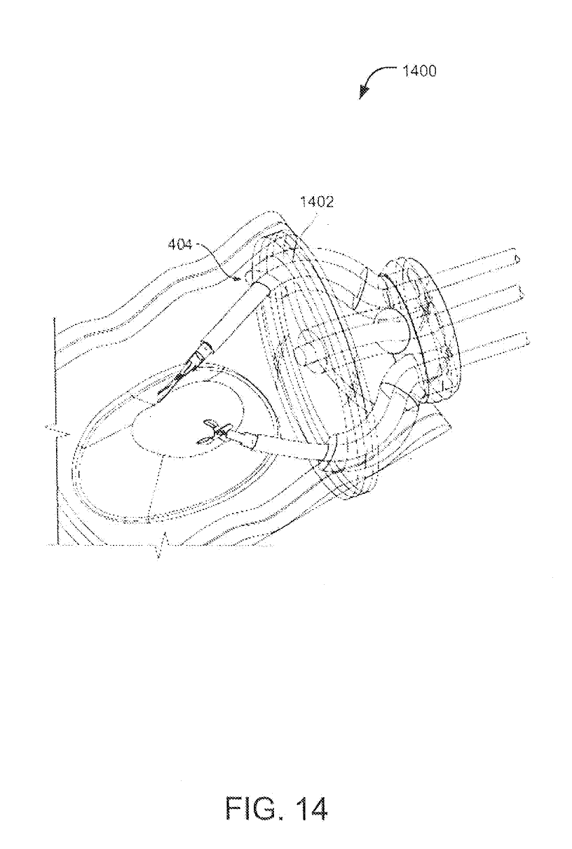

FIG. 14 is a schematic view of another embodiment 1400 of distal end of the guide tube 108, showing an embodiment of the guiding structure 404 that includes a sealing mechanism. As discussed above, the guiding structure 404 may expand once positioned proximate to target tissue 1302. The deployed guiding structure 404 may be configured to form a seal with the surrounding body lumen. The seal may serve to inhibit or prevent the proximal movement of bodily fluids and/or materials through guide tube 108. Likewise, the seal may help prevent the insertion of unwanted materials through guide tube 108. The expansion of the guiding structure 404 may also provide this sealing with the surrounding body parts. As shown, the guiding structure 404 may be manufactured using a self-expanding member 1402 that expands to form a tight seal with a body lumen. Alternatively, the self-expandable member 1402 in the form of a sealant may seal the entire body lumen and it may include apertures for passage of tubular member 410 and/or rail 504. These apertures may also form a seal around the incoming device.

In addition, the expansion may be provided by any conventional expansion mechanism, such as an expandable balloon or control wires 902a, 902b discussed above. Alternatively, a suitable sealing mechanism may include an elastomeric barrier having an expandable opening therein. In addition, any known sealant or adhesive may be applied to the outer surface of the tubular members 410 and/or rails 504.

The present disclosure discloses a system that facilitates insertion of surgical tools to a patient's body through natural or small incision. Further, the tools are appropriately guided towards the target site for desired surgical procedures.

Other embodiments of the disclosure will, be apparent to those skilled in the art from consideration of the specification and practice of the embodiments disclosed herein. It is intended that the specification and examples be considered as exemplary only, with a true scope and spirit of the invention being indicated by the following claims.

What is claimed is:

1. A device for guiding a surgical tool, the device comprising:
a mating section for mating with a portion of a guide tube; and
an elongate structure extending from the mating section, the elongate structure having a proximal end and a distal end, wherein the elongate structure is configured to transition between a collapsed delivery configuration and an expanded deployed configuration.

2. The device of claim 1, wherein the elongate structure includes a plurality of elongate structures, and wherein a distal end of each of the plurality of elongate structure is generally directed at a common location.

3. The device of claim 1, wherein the elongate structure defines a lumen therethrough.

4. The device of claim 1, wherein the elongate structure is a rail for guiding a surgical tool.

5. The device of claim 1, further comprising a triangulation mechanism for moving the elongate structure from the collapsed delivery configuration to the expanded deployed configuration.

6. The device of claim 1, wherein an inner surface of the mating section includes a first geometric configuration configured to mate with a corresponding second geometric configuration on the distal end of the guide tube.

7. The device of claim 1, wherein the mating section is configured to be secured to the distal end of the guide tube through an interference fit.

8. The device of claim 1, wherein the device further includes a sealing mechanism for preventing the flow of bodily materials into the guide tube.

9. The system of claim 1, wherein the elongate structure is self-expandable.

10. A surgical system, comprising:
a proximal portion including a plurality of openings for receiving surgical tools therein;
a guide tube extending distally from the proximal portion, wherein the guide tube defines a plurality of channels corresponding to the openings in the proximal portion; and
a distal portion including a plurality of openings corresponding to the channels defined by the guide tube, wherein the distal portion includes at least one elongate structure, having a proximal end and a distal end, extending distally from at least one of the plurality of openings, wherein the elongate structure is configured to transition between a collapsed delivery configuration and an expanded deployed configuration.

11. The system of claim 10, wherein elongate structure includes a lumen configured to receive a surgical tool therein.

12. The system of claim 10, wherein elongate structure includes a plurality of elongate structures, and when in an expanded configuration, the distal ends of each of the plurality of elongate structures is generally directed at a common location.

13. The system of claim 10, wherein the elongate structure is fixedly secured to the guide tube.

14. The system of claim 10, wherein the proximal portion further includes a control member for selectively manipulating a positioning of the distal portion.

15. The system of claim 10, wherein the at least one of the elongate structure includes a rail for guiding a surgical tool.

16. The system of claim 10, wherein the distal portion is detachably secured to the elongate structure.

17. The system of claim 10, further comprising a triangulation mechanism for moving the elongate structure from the collapsed delivery configuration to the expanded deployed configuration.

18. The system of claim 7, wherein the triangulation mechanism is expandable.

19. The system of claim 10, wherein the at least one elongate structure is self-expandable.

20. A method for performing a surgical procedure at a target site of a body, the method comprising:
advancing a distal portion of a surgical system to a location adjacent the target site, wherein the surgical system comprises:
a proximal portion including a plurality of openings for receiving surgical tools therein, the proximal portion remaining outside of the body;
a guide tube extending distally from the proximal portion, wherein the guide tube defines a plurality of channels; and
the distal portion including a plurality of openings, at least one of the plurality of openings corresponding to one of the plurality of channels defined by the guide tube, wherein the distal portion further includes at least one elongate structure extending distally from the at least one of the plurality of openings, the elongate structure having a proximal end and a distal end, wherein the elongate structure is configured to transition between a collapsed delivery configuration and an expanded deployed configuration;
transitioning the elongate structure from the collapsed delivery configuration to the expanded deployed configuration;
advancing at least one surgical tool from an opening in the proximal portion and through a channel in the guide tube and opening in the distal portion;
guiding the at least one surgical tool to a target location via the elongate structure; and
performing a procedure with the surgical tool.
